(19) United States
(12) Patent Application Publication
(10) Pub. No.: US 2009/0281444 A1
(43) Pub. Date: Nov. 12, 2009

(54) DEVICE FOR SECRETION REMOVAL, MANUAL VENTILATION AND DETERMINATION OF IN VIVO LUNG MECHANICS WITHOUT CIRCUIT DISCONNECTION

(76) Inventor: Robert H. Estetter, City of Garland, TX (US)

Correspondence Address: HITT GAINES P.C., P.O. BOX 832570, RICHARDSON, TX, 75083, US

(21) Appl. No.: 12/437,751

(22) Filed: May 8, 2009

Related U.S. Application Data
(60) Provisional application No. 61/051,389, filed on May 8, 2008.

(51) Int. Cl. A61B 5/087; B23P 11/00

(52) U.S. Cl. 600/538; 29/428

(57) ABSTRACT

An artificial airway obstruction and pulmonary mechanics measuring device comprising a rigid body chamber having a pressure measuring port and a medical ventilator port; a pressure measuring device coupled to the pressure measuring port; and a ventilator port occlusion device coupleable to the medical ventilator port. A method of manufacturing an artificial airway obstruction and pulmonary mechanics measuring device is included.

### CROSS-REFERENCE TO PROVISIONAL APPLICATION

This application claims the benefit of U.S. Provisional Application No. 61/051,389 entitled “DEVICE FOR SECRETION REMOVAL, MANUAL VENTILATION AND DETERMINATION OF IN VIVO LUNG MECHANICS WITHOUT CIRCUIT DISCONNECTION” to Estetter, filed on May 8, 2008 which is commonly owned with the present invention and incorporated herein by reference as if reproduced herein in its entirety.

### TECHNICAL FIELD OF THE INVENTION

The present invention is directed, in general, to a medical device and, more specifically, to a device and method for in vivo secretion removal and manual ventilation of human lungs as well as determination of in vivo lung mechanics without circuit disconnection of a mechanical ventilator.

### BACKGROUND OF THE INVENTION

Currently, in-line lung suction devices allow secretions to be deposited into the ventilator circuit and heat/moisture exchange devices, with the instillation of saline in the lung and resulting coughing by the patient. By isolating the patient from the circuit, saline instillation and secretion removal can be accomplished while minimizing the resultant contamination.

Applying suction to the patient airway that is in communication with the ventilator results in an attempt by the ventilator to deliver a breath. This results in secretions being delivered to the patient rather than being removed. By isolating the patient from the ventilation circuit automatic breath delivery does not occur, thereby facilitating secretion removal.

There currently are no real-time in vivo monitoring methods or devices for determining the optimal lung inflation facilitating sub-tidal volume delivery (Vt_{sub}) during high frequency ventilation (HFV). Decisions are currently made by confirmation of errors in HFV settings through use of arterial blood gases (ABGs) (i.e., principally oxygen and carbon dioxide) measurements, and chest radiography (X-ray) findings. However, results of these tests suffer from lag times averaging 30 minutes to 2 hours post-patient de-compensation. This lack of real-time feedback in HFV management allows for a probability of facilitating ventilator-induced lung injury (VILI) and may cause hemodynamic compromise to the patient secondary to alveolar over-distention affecting cardiac output.

Currently, the patient population receiving HFV is among the most critical in any medical facility. Measurements which require disconnection of the high frequency ventilator allow for de-recruitment of the patient's lungs such that the hysteresis of the lung skews any measurements obtained; thereby resulting in measurements which do not reflect actual conditions. Furthermore, during periods of patient de-compensation it is difficult to rule out pulmonary causes or issues associated with the artificial airway. This delay can result in needless, wrong, or delayed ventilator changes, or unwarranted patient disconnects (i.e., manual ventilation or endotracheal suctioning) that can worsen patient de-compensation. Patient disconnection has also been shown to increase incidence of ventilator-acquired pneumonia (VAP) and patient de-compensation due to lung de-recruitment and cardiac function compromise.

Accordingly, what is needed in the art to facilitate proper setting of the HFV is a device and method that enables determination of optimal lung inflation, as well as the lower and upper inflection points while the patient is connected to the high frequency ventilator.

### SUMMARY OF THE INVENTION

To address the above-discussed deficiencies of the prior art, the present invention provides an artificial airway obstruction and pulmonary mechanics measuring device comprising a rigid body chamber having a pressure measuring port and a medical ventilator port; a pressure measuring device coupled to the pressure measuring port; and a ventilator port occlusion device coupleable to the medical ventilator port. A method of manufacturing an artificial airway obstruction and pulmonary mechanics measuring device is included.

The foregoing has outlined preferred and alternative features of the present invention so that those skilled in the art may better understand the detailed description of the invention that follows. Additional features of the invention will be described hereinafter that form the subject of the claims of the invention. Those skilled in the art should appreciate that they can readily use the disclosed conception and specific embodiment as a basis for designing or modifying other structures for carrying out the same purposes of the present invention. Those skilled in the art should also realize that such equivalent constructions do not depart from the spirit and scope of the invention.

### BRIEF DESCRIPTION OF THE DRAWINGS

For a more complete understanding of the present invention, reference is now made to the following descriptions taken in conjunction with the accompanying drawings, in which:

FIG. 1A illustrates a functional block diagram of a patient's lung on a conventional high frequency oscillatory ventilator (HFOV) as in the prior art;

FIG. 1B illustrates a functional block diagram of a patient's lung on a conventional high frequency oscillatory ventilator (HFOV) equipped with an artificial airway tube obstruction and pulmonary mechanics measuring device constructed according to the principles of the present invention;

FIG. 2A illustrates a sectional view of one embodiment of the artificial airway obstruction and pulmonary mechanics measuring device of FIG. 1;

FIG. 2B illustrates a sectional view of the pulmonary mechanics measuring device of FIG. 1 without the medical ventilator port occluded and the inline suction catheter advanced allowing secretion removal as is currently practiced;

FIG. 2C illustrates a sectional view of the pulmonary mechanics measuring device of FIG. 1 with the medical ventilator port occluded;

FIG. 2D illustrates a sectional view of the pulmonary mechanics measuring device of FIG. 1 with the standard medical ventilator occluded and the suction catheter advanced for suctioning from the lung;

FIG. 2E illustrates an isometric view of an alternative embodiment of a sliding sleeve incorporating a twist locking mechanism;

FIG. 2F illustrates a sectional view of a first alternative embodiment of a pulmonary mechanics measuring device employing the sliding sleeve of FIG. 2E locked in the open position;

FIG. 2G illustrates a sectional view of the measuring device of FIG. 2F with the sleeve locked forward thereby interrupting ventilation while isolating the endotracheal tube and patient lungs;

FIG. 2H illustrates a sectional view of an alternative embodiment of the rigid body chamber of FIG. 2F with the sliding sleeve locked in the open position;

FIG. 2I illustrates a sectional view of a second alternative embodiment of the rigid body chamber of FIG. 2F with the sliding sleeve locked forward thereby occluding the medical ventilator port and enabling secretion removal with manual ventilation using manual resuscitation bag while avoiding circuit contamination and ventilator interference;

FIG. 3A illustrates a sectional view of a second alternative embodiment of a pulmonary mechanics measuring device constructed according to the principles of the present invention;

FIG. 3B illustrates a sectional view of the pulmonary mechanics measuring device of FIG. 3A with the flexible balloon catheter advanced past the hub and into the standard endotracheal tube; thus, allowing for brief interruption (<15 seconds) of mechanical breaths and injection of a known volume from the volume delivery device to the human lung;

FIGS. 4A-4D illustrate sectional views of a third embodiment of a pulmonary mechanics measuring device comprising a rotating valve assembly;

FIGS. 5A and 5B illustrate sectional views of a fourth embodiment of a pulmonary mechanics measuring device constructed according to the principles of the present invention;

FIGS. 6A and 6B illustrate sectional views of a fifth embodiment of a pulmonary mechanics measuring device and elevation views of a gate valve constructed according to the principles of the present invention;

FIG. 7 illustrates a plan view of a sixth embodiment of a pulmonary mechanics measuring device constructed according to the principles of the present invention;

FIG. 8 illustrates a plan view of a seventh embodiment of a pulmonary mechanics measuring device constructed according to the principles of the present invention;

FIG. 9 illustrates the procedural steps necessary to obtain pulmonary mechanics measurements for determination of proper HFV settings;

FIG. 10 illustrates a chart to convert pressure measurements to compliance; and

FIG. 11 illustrates an example of the relationship of lung hysteresis and Vt_{sub} (sub-tidal volume) delivery during high frequency oscillatory ventilation (HFOV).

### DETAILED DESCRIPTION

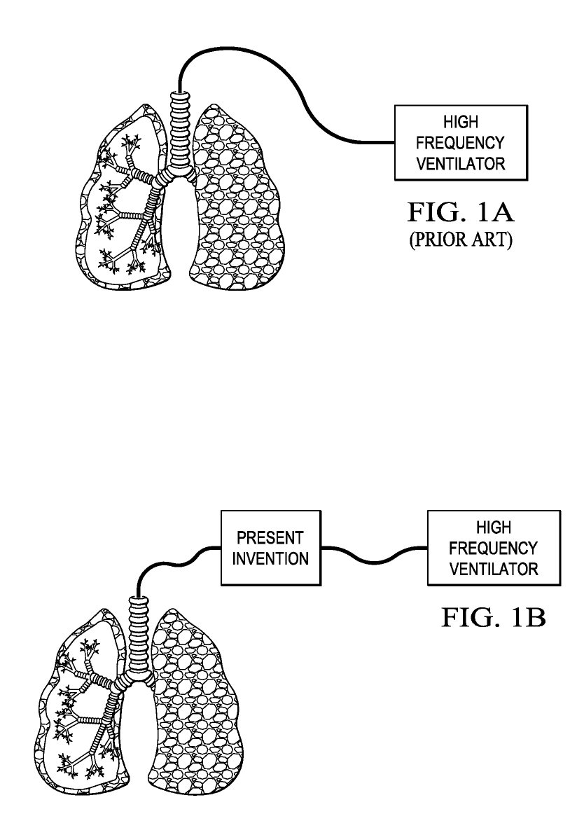

Referring initially to FIGS. 1A and 1B, illustrated are functional block diagrams of a patient's lung on a conventional high frequency oscillatory ventilator (HFOV) as in the prior art and the same patient's lung on the conventional high frequency oscillatory ventilator equipped with an artificial airway obstruction and pulmonary mechanics measuring device 100 constructed according to the principles of the present invention, respectively.

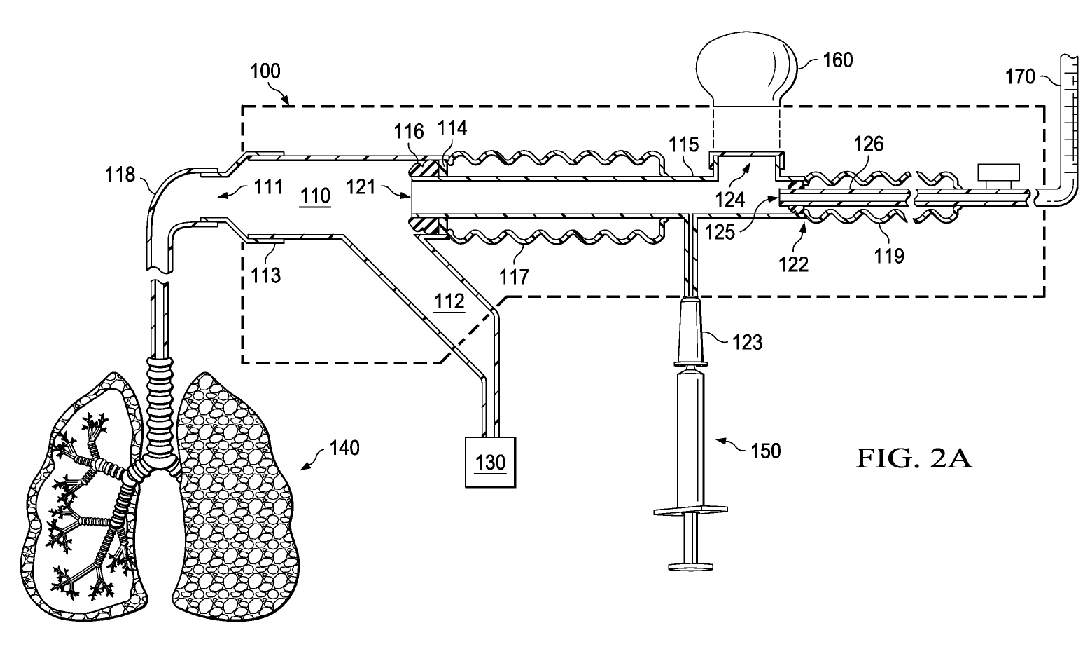

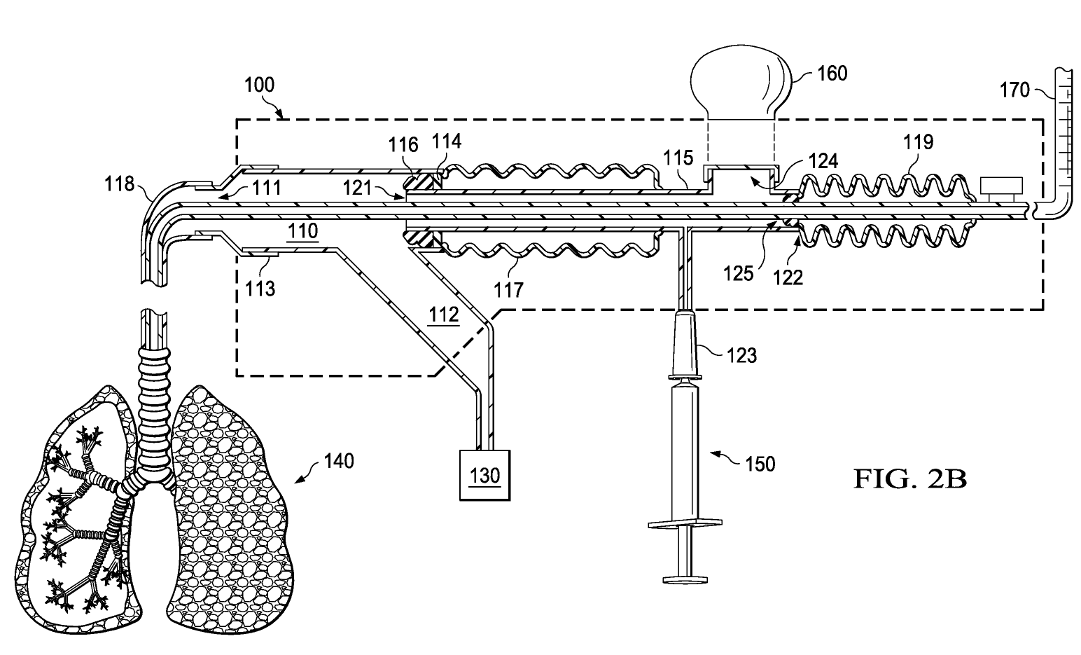

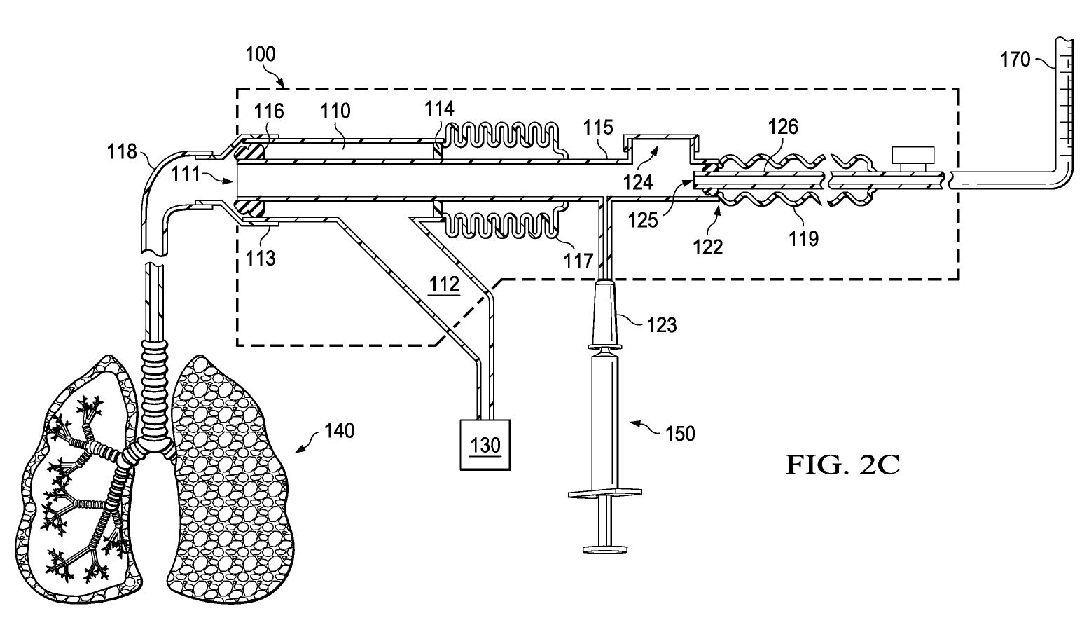

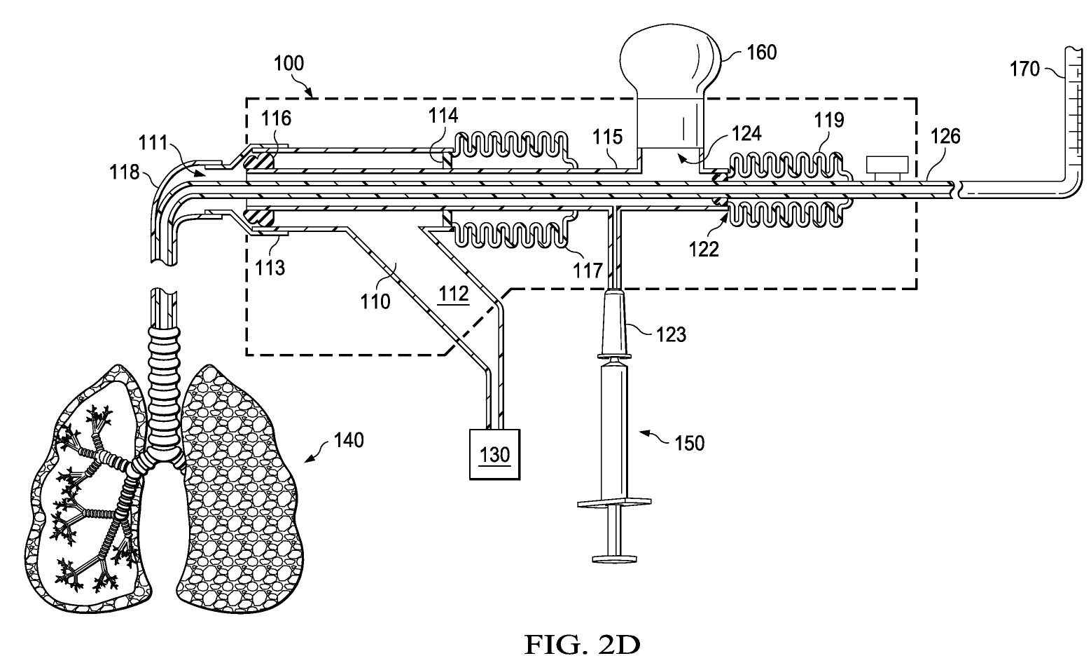

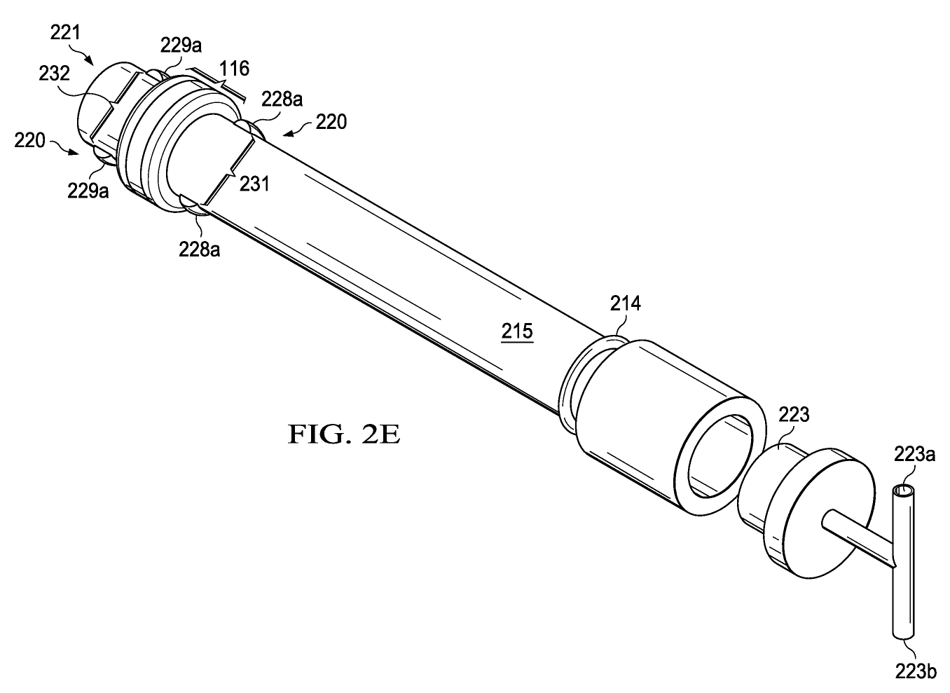

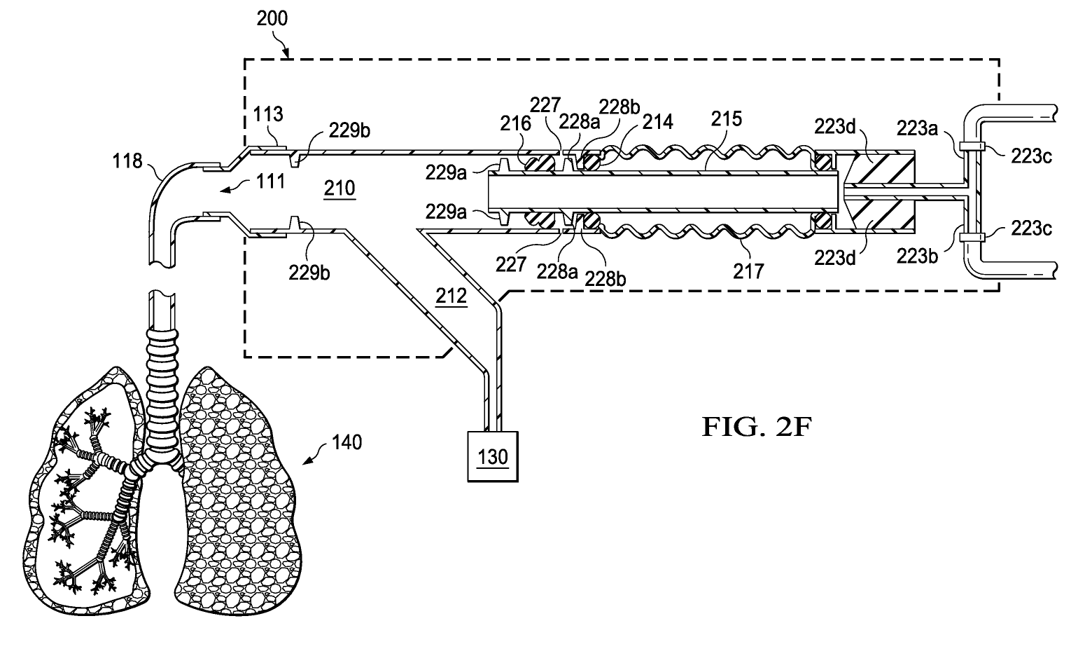

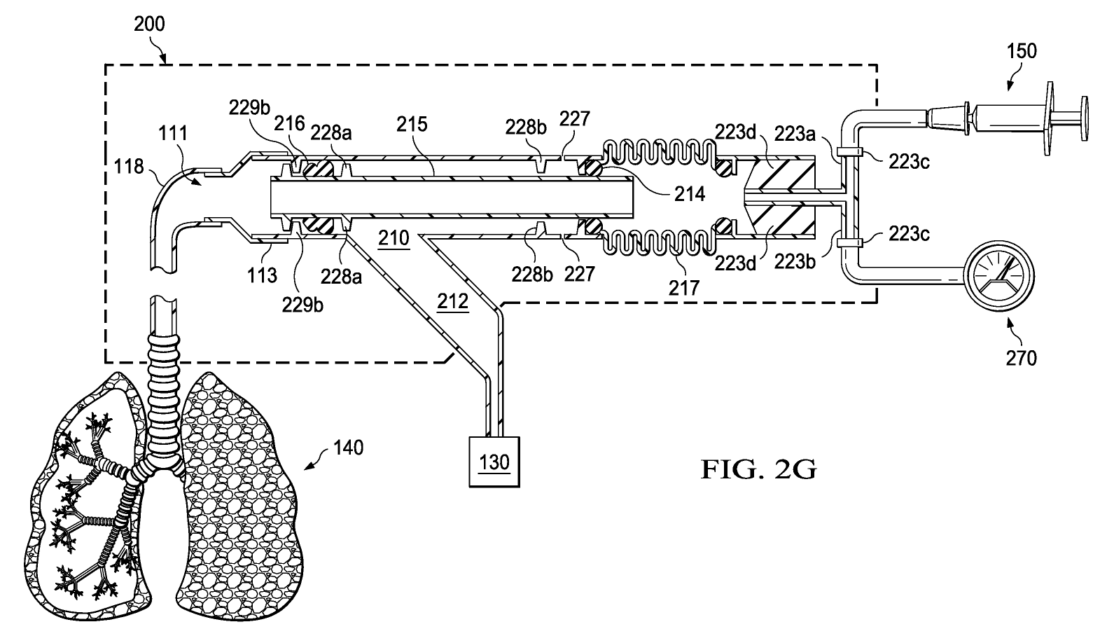

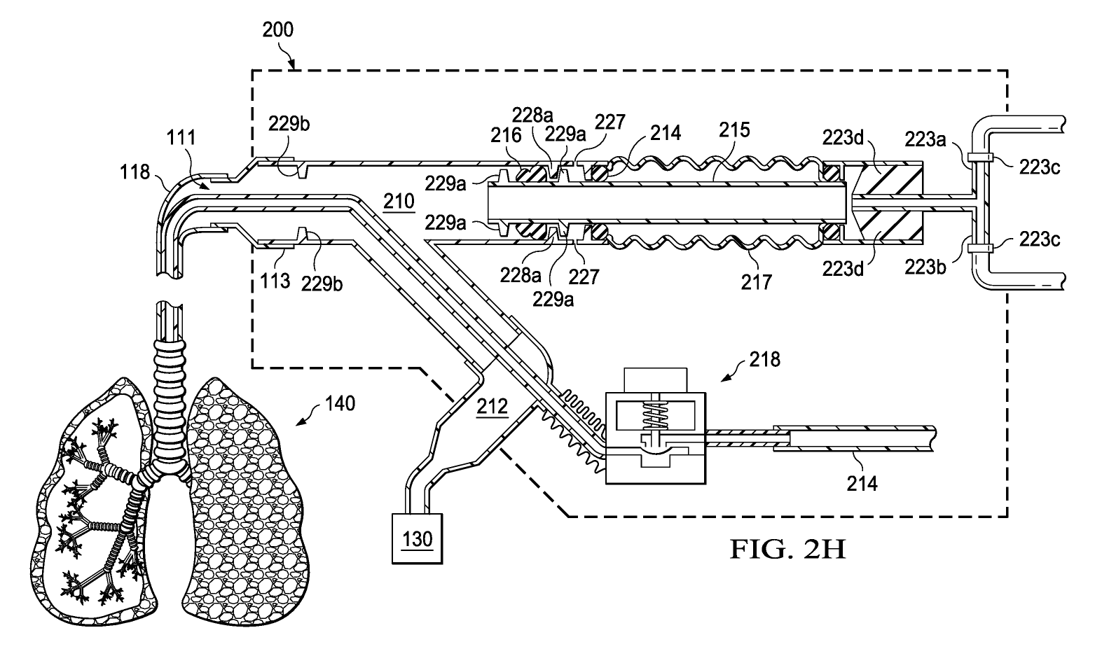

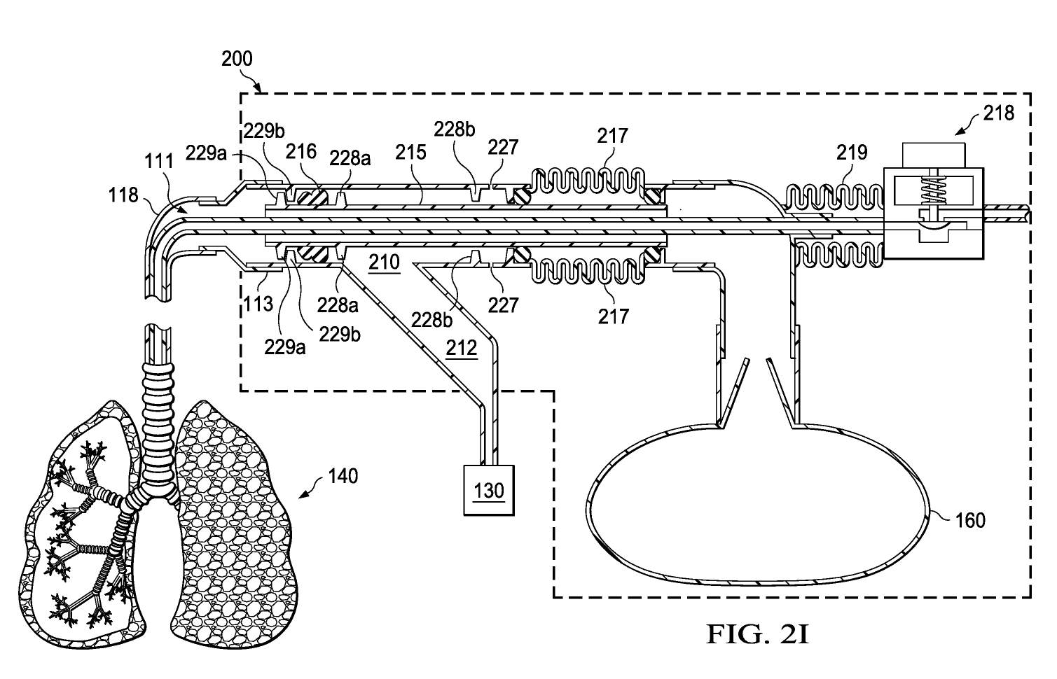

Referring now to FIG. 2A, illustrated is a sectional view of one embodiment of the artificial airway obstruction and pulmonary mechanics measuring device 100 of FIG. 1. For the purposes of this discussion, the artificial airway obstruction and pulmonary mechanics measuring device 100 will henceforth be referred to as the compliance measuring device 100. The compliance measuring device 100 comprises a rigid body chamber 110 having an endotracheal tube (ETT) port 111 and a medical ventilator port 112; at least one O-ring seal 114; a hollow, slidable, pressure measuring stem 115; a pliable plunger 116; first, second, and third ports 123, 124, 125, respectively; first and second protective sheaths 117, 119, respectively; and an in-line suction catheter 126. The rigid body chamber 110 is coupleable to a standard medical ventilator circuit 130 at the medical ventilator port 112 and is further coupleable to a hub 113 of standard 15 mm endotracheal tube 118 at ETT port 111 allowing for uninterrupted ventilation of a patient's lung 140 via the standard medical ventilator 130 coupled to the medical ventilator port 112. In the embodiment of FIG. 2A, at least a portion of the pressure measuring stem 115 is enclosed in the first protective sheath 117. The pliable plunger 116 is coupled to the pressure measuring stem 115 at a first end 121 of the stem 115 and branches into the first, second, and third ports 123, 124, 125, respectively, at the second end 122 of the stem 115. The first port 123 is used as: a saline injection port, a pressure measuring port, or a volume injection port. When used as a volume injection port 123, a known volume of air is injected into the pressure measuring stem 115 and consequently the lung 140 from a volume delivery device 150. The volume delivery device 150 may be a syringe, a bellows, or other device where a precise volume of air can be measured and injected through the first port 123. The second port 124 enables coupling with a manual resuscitation bag 160 for facilitating manual ventilation when not obtaining pressure measurements. The third port 125 enables introduction of the inline suction catheter 126 which remains withdrawn as shown and in the closed position during measurement maneuvers. The inline suction catheter 126 is enclosed in the second protective sheath 119. A plateau pressure measurement is collected from a manometer 170, pressure gauge, or other pressure sensing device before instilling the known volume.

Referring now to FIG. 2B, illustrated is a sectional view of the pulmonary mechanics measuring device 100 of FIG. 1 without the medical ventilator port 112 occluded and the inline suction catheter 126 advanced allowing secretion removal as is currently practiced.

Referring now to FIG. 2C, illustrated is a sectional view of the pulmonary mechanics measuring device 100 of FIG. 1 with the medical ventilator port 112 occluded. The sliding pressure measuring stem 115 and the pliable plunger 116 are briefly advanced during the testing maneuver into the position shown. The pliable plunger 116 occludes the medical ventilator port 112 when advanced inside of the hub 113 of the standard endotracheal tube 118, thus allowing for a brief interruption, i.e., <5 seconds, of mechanical breaths. A starting pressure as well as pressure after injection of the known volume from the volume delivery device 150 to the human lung 140 is collected from the manometer 170 or other pressure sensing device coupled to port 125. The compliance of the lung is then calculated by dividing the difference in pressures (before and after volume injection, or delta pressure) into the known delivered volume and is reported as cc/cm_{H2O}.

Referring now to FIG. 2D, illustrated is a sectional view of the pulmonary mechanics measuring device 100 of FIG. 1 with the standard medical ventilator 130 occluded and the suction catheter 126 advanced for suctioning from the lung 140. In this position, the medical ventilator 130 is temporarily prevented from ventilating the patient and the port 124 may be attached to a bag valve 160 device for manual ventilation while port 123 may be used for introducing saline solution into the lung 140 which is then removed via the suction catheter 126.

Referring now to FIG. 2E, illustrated is an isometric view of an alternative embodiment of a sliding sleeve 215 incorporating a twist locking mechanism 220. The twist locking mechanism 220 comprises a plurality of lugs 228a, 229a extending outwardly from and proximate a first end 221 of the sliding sleeve 215. As can be seen, the plurality of lugs 228a, 229a extend partially circumferentially around an outer surface of the sliding sleeve 215, there being circumferential gaps 231, 232 between the lugs 228a, 229a. The sliding sleeve 115 further comprises a removable plug 223 configured to allow volume introduction/removal through orifice 223a while pressure is monitored through 223b. These modifications allow twist locking the mechanism forward within the rigid body chamber 210 allowing airway isolation while facilitating secretion removal. (See FIG. 2F) An additional twist locking mechanism 228 allows the sleeve 215 to be locked in the open position allowing mechanical ventilation to be resumed unimpeded.

Referring now to FIG. 2F, illustrated is a sectional view of a first alternative embodiment of a pulmonary mechanics measuring device 200 employing the sliding sleeve of FIG. 2E locked in the open position. Both the male and female twist locks of 229 and 228 can be seen in this view. The rigid body chamber 210 further comprises at least two alarm whistling orifices 227 and an O-ring seal 214. Also shown in this embodiment are the monitoring orifice caps 223c. A plurality of cooperating lugs 228b, 229b extend inwardly from and proximate opposite ends of the rigid body chamber 210. In a like manner, the plurality of cooperating lugs 228b, 229b extend partially circumferentially around an inner surface of the rigid body chamber 210. The sliding sleeve 215 may be withdrawn when the lugs 229a are aligned with the gaps and withdrawn to a position such that lugs 228a are to the right of cooperating lugs 228b, and the sliding sleeve 215 rotated to a locked position with the lugs 228a aligned with the cooperating lugs 228b. When the sliding sleeve 215 is locked open (to the right) as in FIG. 2F, the compliance measuring device 200 is set for ventilation.

Referring now to FIG. 2G, illustrated is a sectional view of the pulmonary mechanics measuring device of FIG. 2F with the sleeve 215 locked forward thereby interrupting ventilation while isolating the endotracheal tube 118 and the patient's lungs 140. Aligning the lugs 228a with gaps between the cooperating lugs 228b allows the sliding sleeve 215 to be advanced (to the left in the FIGURE) until the lugs 229a align with gaps between the cooperating lugs 229b. Once the sliding sleeve 215 is advanced so that the lugs 229a are to the left of the cooperating lugs 229b, the sliding sleeve 215 may be rotated to prevent the sliding sleeve 215 from extending to the right under pressure. When the sliding sleeve 215 is locked to the left as in FIG. 2G, the medical ventilator port 212 is occluded and the sliding sleeve 215 is positioned for volume insertion/removal and pressure measurements. The two alarm whistling orifices 227 are actively driven by the mechanical ventilator pressure generated. This alarm is active whenever the sleeve 215 is not in the locked open position indicating that the patient is not being ventilated. The measuring cap 223d is connected through 223a to the measuring syringe 150 for introduction and removal of known volumes. Pressure changes are measured through a monitoring device, i.e., a gauge 270 attached to port 223b.

Referring now to FIG. 2H, illustrated is a sectional view of an alternative embodiment of the rigid body chamber 210 of FIG. 2F with the sliding sleeve 215 locked in the open position. This setup allows an in-line suction catheter 214 connected to the measuring device 100 at ventilator interface 212 thereby allowing conventional secretion removal during mechanical ventilation. Alternatively, the sliding sleeve 215 may be locked forward to occlude the medical ventilator port 212 for lung mechanics determination. A valve 218 controls opening or closing the in-line suction catheter 214.

Referring now to FIG. 2I, illustrated is a sectional view of a second alternative embodiment of the rigid body chamber 210 of FIG. 2F with the sliding sleeve 215 locked forward thereby occluding the medical ventilator port 212 and enabling secretion removal with manual ventilation using manual resuscitation bag 160 while avoiding circuit contamination and ventilator interference.

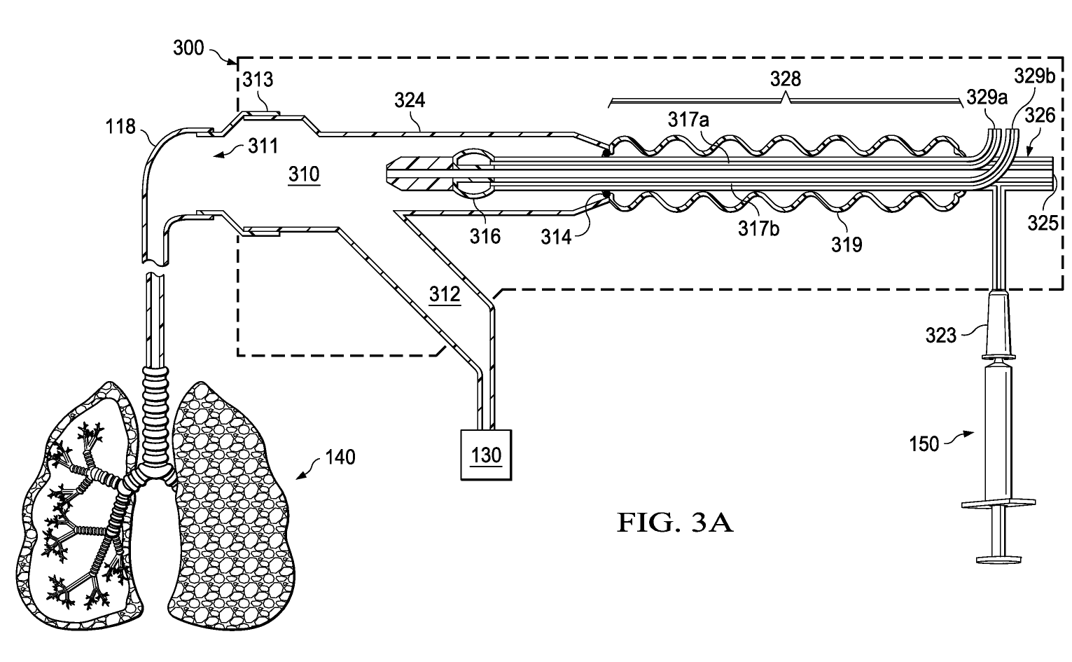

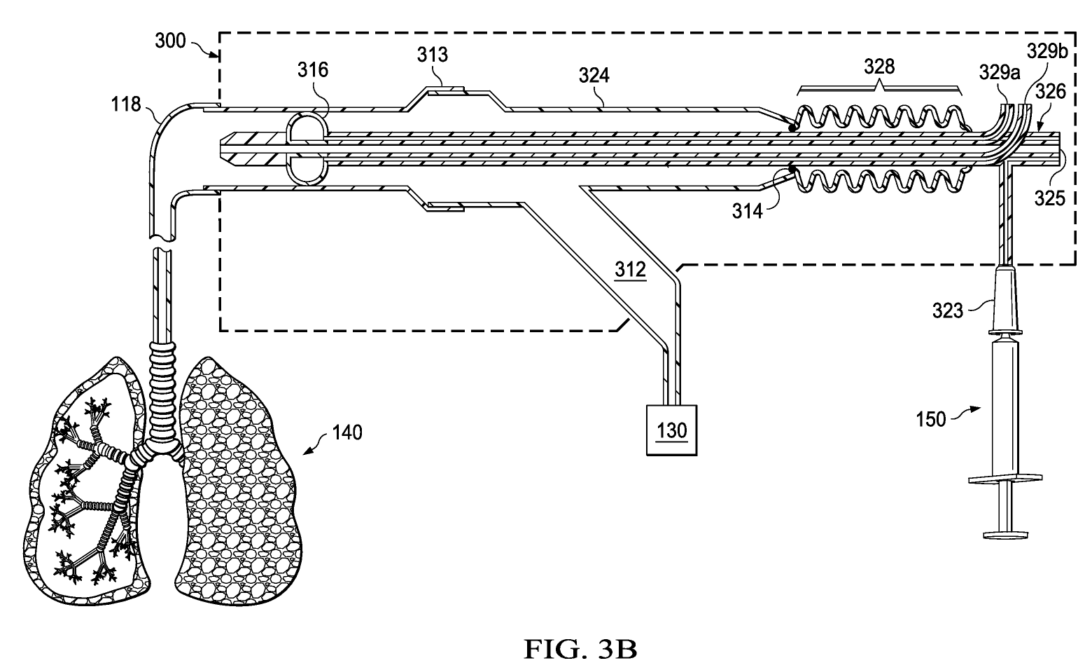

Referring now to FIG. 3A, illustrated is a sectional view of a second alternative embodiment of a pulmonary mechanics measuring device 300 constructed according to the principles of the present invention. The compliance measuring device 300 comprises a rigid body chamber 310; at least one O-ring seal 314; a protective sheath 319; a volume injection port 323; a fixed sleeve 324; a manometer port 325; a flexible balloon catheter 328; a hollow, slidable, pressure measuring stem 326; and a cuff inflation port 329. The rigid body chamber 310 comprises an endotracheal tube (ETT) port 311 and a medical ventilator port 312. The rigid body chamber 320 attaches to a standard medical ventilator 130 through the medical ventilator port 312 and to the hub 313 of a standard 15 mm endotracheal tube 118. The fixed sleeve 324 houses the flexible balloon catheter 328 which is seated and sealed by the O-ring 314. The balloon catheter 328 comprises an inflatable/deflatable cuff 316, first and second channels 317a, 317b, one used to inflate or deflate the cuff 316 by a volume of gas injected via the cuff inflation/deflation ports 329a, 329b and the other orifice 323 is used to inject a known volume via the volume delivery device 150 thru the volume injection port 323 and obtain pressure measurements from the manometer port 325. The complete balloon catheter 328 is enclosed and protected from the outside environment by the flexible protective sleeve 319.

Referring now to FIG. 3B, illustrated is a sectional view of the pulmonary mechanics measuring device 300 of FIG. 3A with the flexible balloon catheter 326 advanced past the hub 313 and into the standard endotracheal tube 118; thus, allowing for brief interruption (<15 seconds) of mechanical breaths and injection of a known volume from the volume delivery device 150 to the human lung 140. A plateau pressure measurement is collected from the pressure measurement port 325 before instilling the known volume and again after injecting the known volume. The compliance of the lung is calculated by dividing the change in pressure measured (delta pressure) into the known tidal volume and reported as cc/cm_{H2O}.

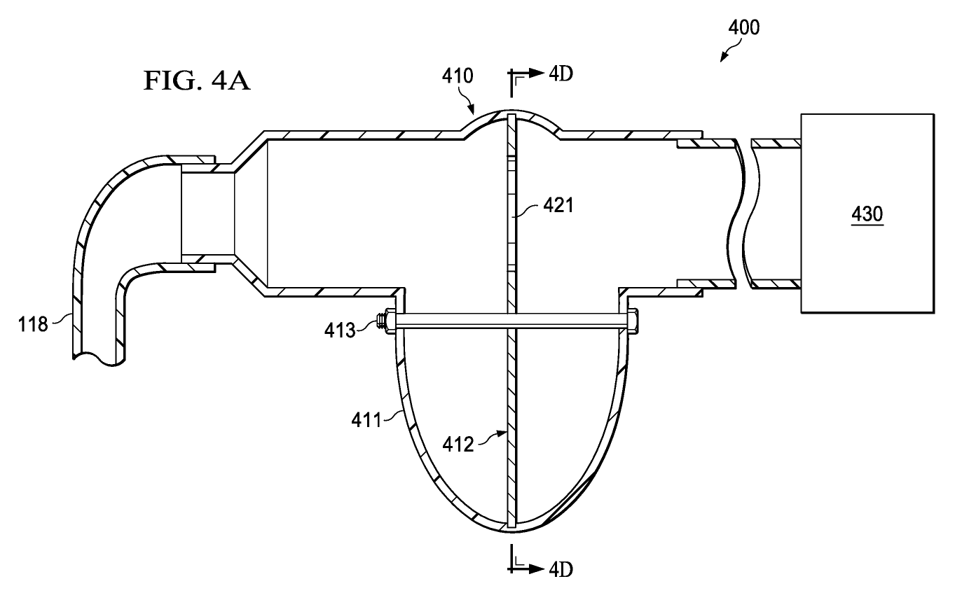

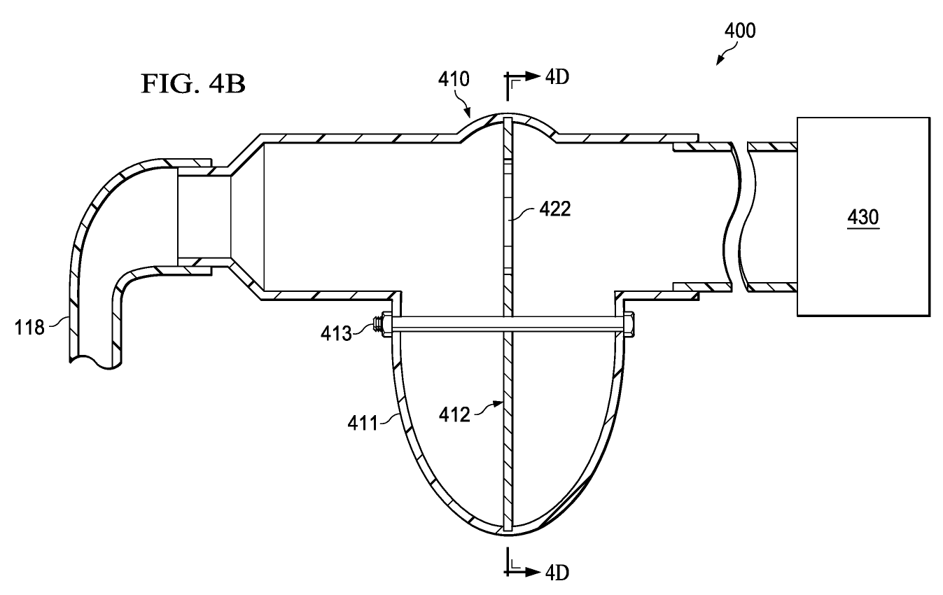

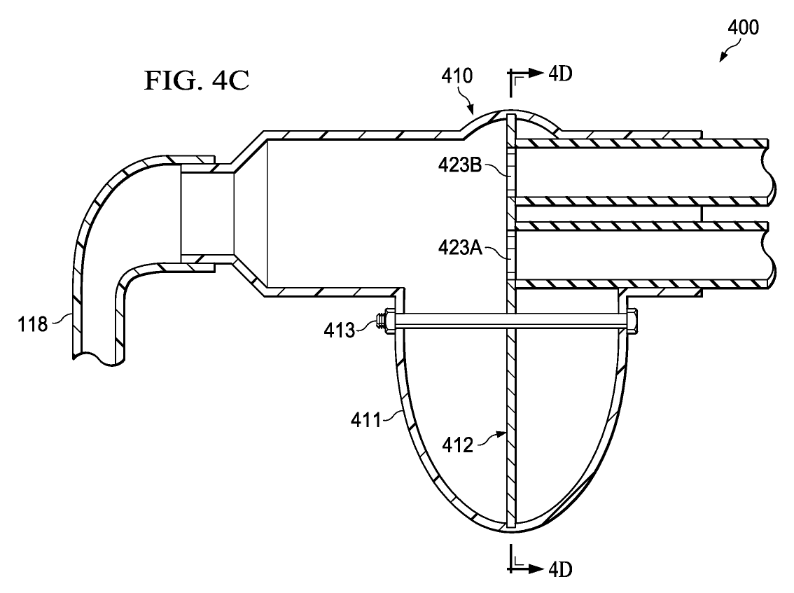

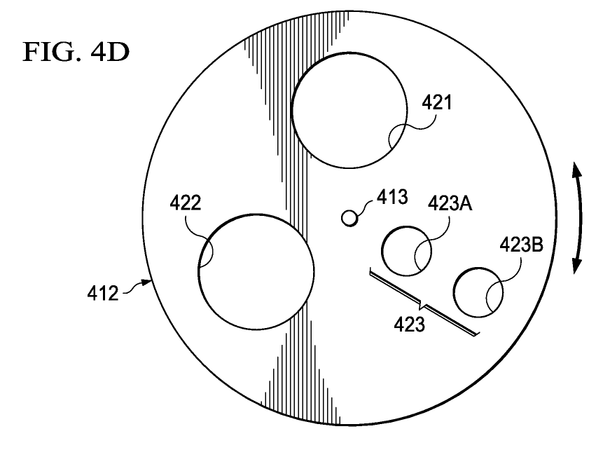

Referring now to FIGS. 4A-4D, illustrated are sectional views of a third embodiment of a pulmonary mechanics measuring device 400 comprising a rotating valve assembly. The compliance measuring device 400 comprises a valve assembly 410 used to control communication with the standard endotracheal tube 118. The valve assembly 410 comprises at least one circular shaped rigid body 411, a rotatable plate 412 and a solid pin assembly 413. The rotatable plate 412 has a plurality of orifices 421-423 therethrough and rotates around the solid pin assembly 413. The plurality of orifices 421-423 are first, second and third positional ports 421-423, respectively. The first positional port 421 (FIG. 4A) is a ventilation port which enables uninterrupted ventilation to the patient when positioned inline of the in-patient ventilator circuitry (endotracheal tube 118) by means of ventilator 430. The second positional port 422 is a suction port enabling endotracheal suctioning when positioned inline of the in-patient ventilator circuitry. The third positional port 423 comprises first and second ports, 423a, 423b wherein the first port 423a is a volume injection port used to instill a volume of gas when positioned inline of the in-patient ventilator circuitry, and the second port 423b is a pressure measuring port used to capture pressure measurements when positioned inline of the in-patient ventilator circuitry. FIG. 4B illustrates a sectional view of the rotating valve in the ventilation position allowing access for manual bag valve ventilation, saline instillation and endotracheal suctioning. FIG. 4C illustrates a sectional view of the rotating valve in the measurement position allowing the instillation of a volume of gas to the patient and the taking of pressure measurements at the pressure measuring port 423b.

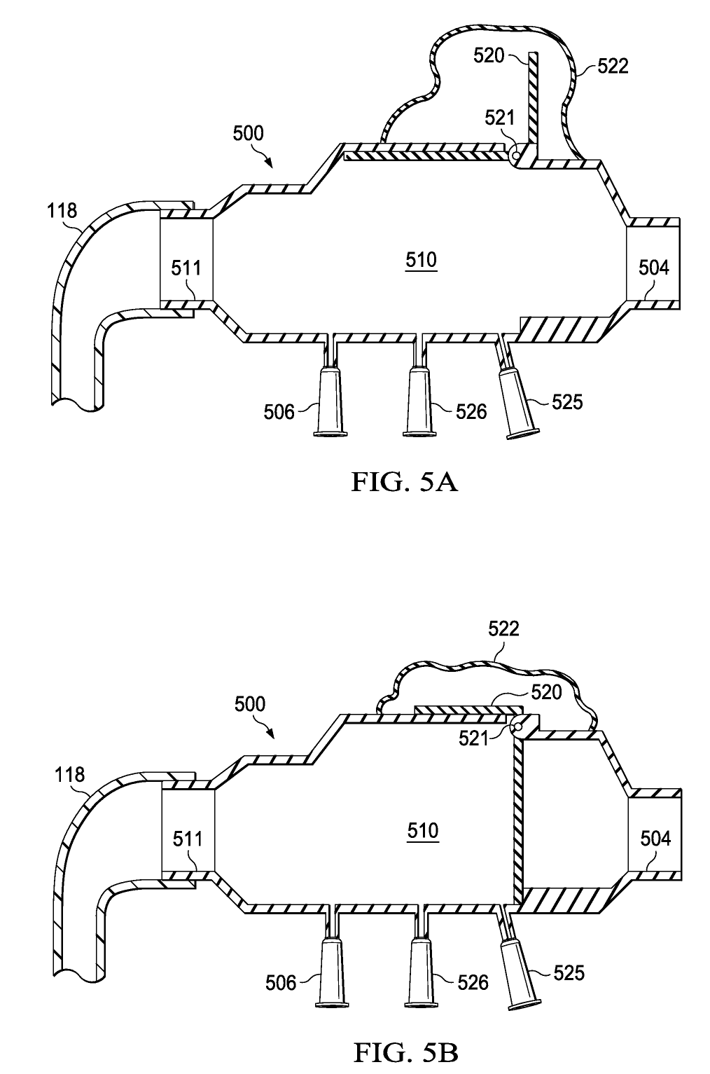

Referring now to FIGS. 5A and 5B, illustrated are sectional views of a fourth embodiment of a pulmonary mechanics measuring device 500 constructed according to the principles of the present invention. The pulmonary mechanics measuring device 500 comprises a rigid body chamber 510, a ventilation port 504, an ETT port 511, and a hinged valve assembly 520. In this embodiment, the valve assembly 520 is hinged at a point 521 so that the valve will rotate into an obstructed position as shown in FIG. 5B thereby occluding ventilation by the HFV (not shown). A flexible sleeve 522 envelops the valve assembly 520. Ventilation port 504 couples to the HFV (not shown) and provides ventilation when not obstructed by the hinged valve 520. The patient's endotracheal tube 118 is coupled at ETT port 511. Port 526 may be used for measuring pressure as described above. Port 506 may also be fluidly coupled to port 526 or simply in addition to port 526 to provide a means for instilling a known volume of gas as described above. The rigid body chamber 510 may be shaped so as to provide port 525 with a more direct access for insertion of a flexible catheter into the rigid body chamber 510 and on into the ETT 118. The valve 520 may be opened and closed either manually or with a solenoid.

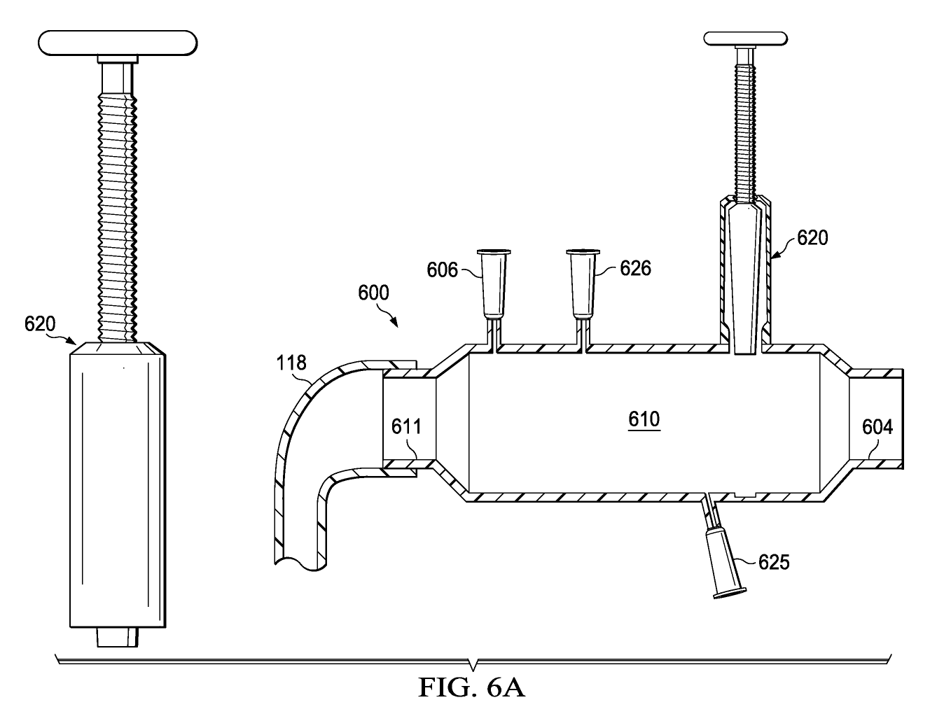

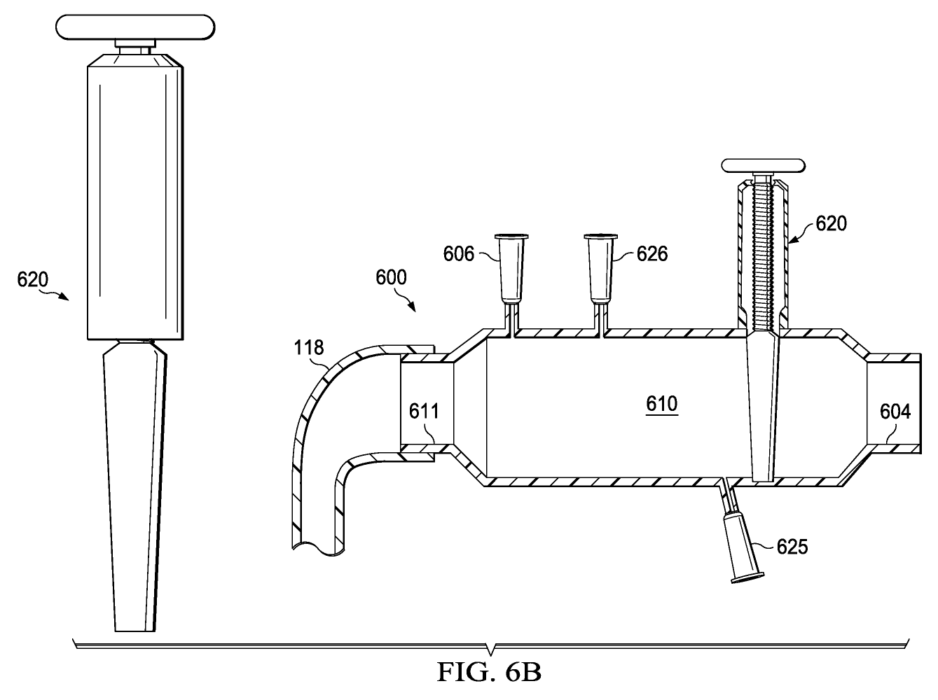

Referring now to FIGS. 6A and 6B, illustrated are sectional views of a fifth embodiment of a pulmonary mechanics measuring device 600 and elevation views of a gate valve constructed according to the principles of the present invention. The pulmonary mechanics measuring device 600 comprises a rigid body chamber 610, a ventilation port 604, an ETT port 611, and a gate valve assembly 620. The gate valve 620 may be either manual or solenoid operated. Ventilation port 604 couples to the HFV (not shown) and provides ventilation when not obstructed by the gate valve 620. The patient's endotracheal tube 118 is coupled at ETT port 611. When gate valve 620 is closed, port 626 may be used for measuring pressure as described above. Port 606 may also be fluidly coupled to port 626 or simply in addition to port 626 to provide a means for instilling a known volume of gas as described above. The rigid body chamber 610 may be shaped so as to provide port 625 with a more direct access for insertion of a flexible catheter into the rigid body chamber 610 and on into the ETT 118.

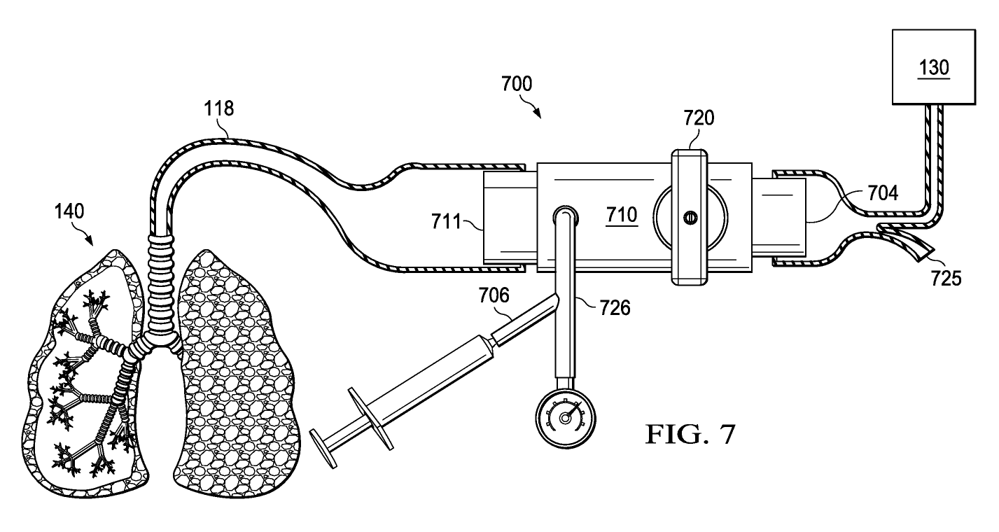

Referring now to FIG. 7, illustrated is a plan view of a sixth embodiment of a pulmonary mechanics measuring device 700 constructed according to the principles of the present invention. The pulmonary mechanics measuring device 700 comprises a rigid body chamber 710, a ventilation port 704, an ETT port 711, and a single ball valve assembly 720. The single ball valve 720 may be either manual or solenoid operated. Ventilation port 704 couples to the HFV 130 and provides ventilation when not obstructed by the single ball valve 720. The patient's endotracheal tube 118 is coupled at ETT port 711. When single ball valve 720 is closed, port 726 may be used for measuring pressure as described above. Port 706 may also be fluidly coupled to port 726 or simply in addition to port 726 to provide a means for instilling a known volume of gas as described above. The rigid body chamber 710 may be shaped so as to provide port 725 with a more direct access for insertion of a flexible catheter into the rigid body chamber 710 and on into the ETT 118.

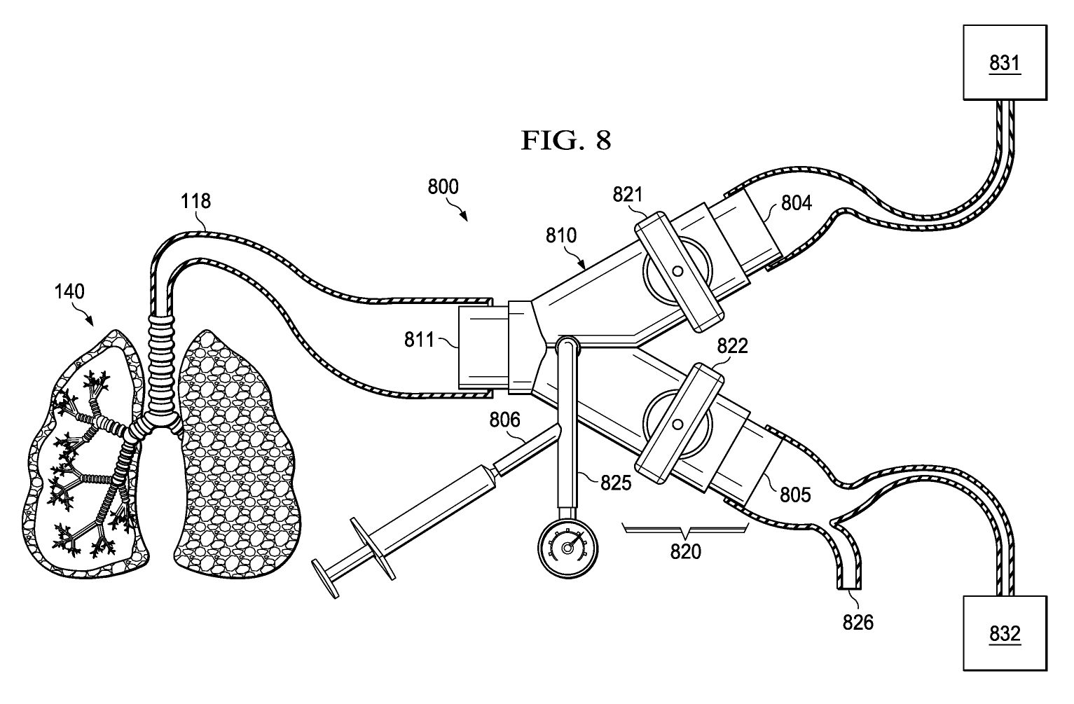

Referring now to FIG. 8, illustrated is a plan view of a seventh embodiment of a pulmonary mechanics measuring device 800 constructed according to the principles of the present invention. The pulmonary mechanics measuring device 800 comprises a rigid body chamber 810, first and second ventilation ports 804, 805, an ETT port 811, and a double ball valve assembly 820. The double ball valve assembly 820 comprises a first ball valve 821 and a second ball valve 822 and either valve may be manual or solenoid operated. The first and second ventilation ports 804, 805, respectively, may be coupled to first and second HF ventilators 831, 832 or one ventilation port 804 may be coupled to one ventilator and the other ventilation port 805 may be available to a suction catheter 826. The HFV 831 provides ventilation when not obstructed by the first ball valve 821. The patient's endotracheal tube 118 is coupled at ETT port 811. When the first ball valve 821 is closed, ventilator 831 is isolated and suction catheter 826 may be fed through port 805 and then on into the ETT 118. Port 826 may be used for measuring pressure as described above. Alternatively, port 825 may be used for measuring pressure and port 806 may be used for instilling a known volume of gas as described above. The Y-valve described above using ball valves on both limbs may be used to collect measurements, change ventilators without disconnection, alternate vent modes, secretion clearance, etc.

Referring now to Table 1, illustrated are the advantages of the various valve configurations.

| | | Y | | Y | Single | Balloon | | Single |
|---|---|---|---|---|---|---|---|---|
| | Rotary | Ball | Plunge | Gate | Gate | Catheter | Swing | Ball |
| Advantages | Valve | Valve | Valve | Valve | Valve | Valve | Valve | Valve |
| Static Compliance | Yes | Yes | Yes | Yes | Yes | Yes | Yes | Yes |
| Determination | | | | | | | | |
| Dynamic | Yes | Yes | Yes | Yes | Yes | Yes | Yes | Yes |
| Compliance | | | | | | | | |
| Determination | | | | | | | | |
| Artificial Airway | Yes | Yes | Yes | Yes | Yes | Yes | Yes | Yes |
| Resistance changes | | | | | | | | |
| PEEP Inflection | Yes | Yes | Yes | Yes | Yes | Yes | Yes | Yes |
| Point Determination | | | | | | | | |
| Upper Inflection | Yes | Yes | Yes | Yes | Yes | Yes | Yes | Yes |
| Point Determination | | | | | | | | |
| Ventilation mode | Yes | Yes | Yes | Yes | No | No | No | No |
| change without | | | | | | | | |
| disconnect | | | | | | | | |
| Conventional | Yes | Yes | Yes | Yes | No | No | No | No |
| medication delivery | | | | | | | | |
| Bag valve suction | Yes | Yes | Yes | Yes | No | No | No | No |
| without disconnect | | | | | | | | |

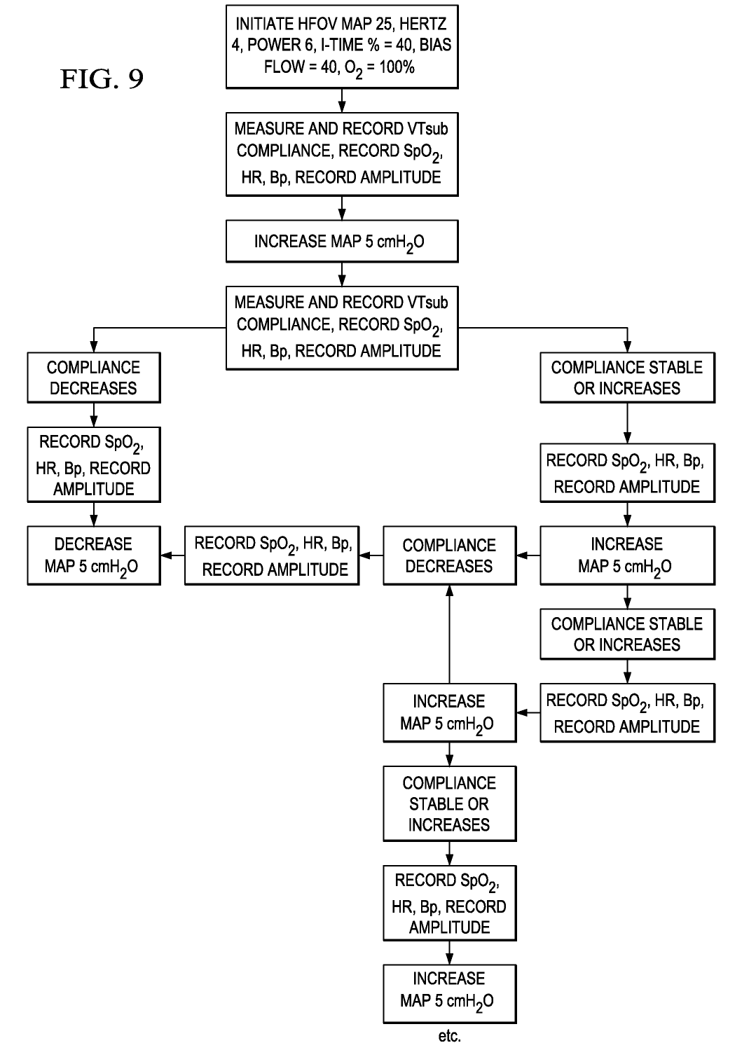

Referring now to FIG. 9, illustrated are the procedural steps necessary to obtain compliance measurements for determination of proper HFV settings. The steps are: (1) the patient is on the HFV and is ventilating; (2) the patient is isolated from the HFV using a valve, a balloon, or a plunger; (3) the patient is isolated and the lung pressure is read; (4) the patient is in fluid communication with a syringe, bulb, or bellows whereby a known volume is injected; and (5) the lung pressure is read again; (6) the volume is let out; and (7) the valve, balloon, or plunger is removed to open the airway back up to the HFV. The patient is then ventilating again with the assistance of the ventilator without breaking the circuit or exposing the patient to potential problems. The entire maneuver takes about three to four seconds, so there is very little likelihood that anything is going to harm the patient.

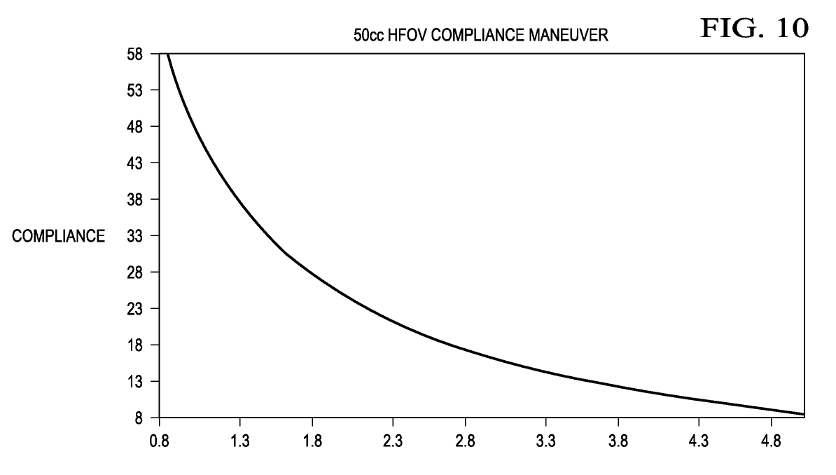

Referring now to FIG. 10, illustrated is a chart to convert pressure measurements to compliance. FIG. 10 saves the technician from having to do the mathematics to determine compliance. Enter the abscissa with the pressure change and read the compliance on the ordinate. For example, enter the abscissa with the pressure change, i.e., pressure after injecting the air volume minus the pressure before injecting the air volume. Thus, if there is a pressure change of 5 centimeters of water pressure for 50 cc of air volume, one can read on the left side of the chart that the compliance is 10. If the pressure change is 1, one can see that the compliance is 50.

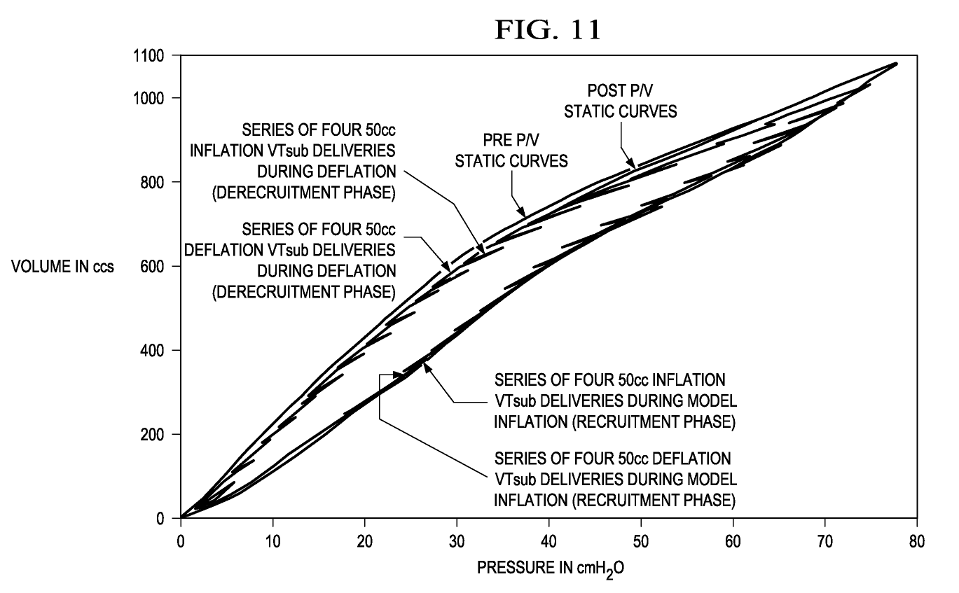

Referring now to FIG. 11, illustrated is an example of the relationship of lung hysteresis and Vt_{sub} (sub-tidal volume) delivery during high frequency oscillatory ventilation (HFOV). In an adult, a proper injected volume is between about 50 cc and 100 cc (normal Vt_{sub} in adult). FIG. 11 shows where the 50 cc tidal volumes are actually being delivered during high frequency ventilation. The static pressure/volume curves are first generated with the slow flow method. Controlled volumes are injected increasing pressure thereby changing lung conditions are exhibited on the lower (recruitment) solid curve. Then, controlled volumes are injected during decreasing lung pressure are exhibited on the upper (de-recruitment) solid curve. The illustrated charts are static pressure/volume curves. The procedure is repeated using 4×50 cc isolated oscillations for each inflation and deflation step. The results show that Vt_{sub} delivery is lung phase dependent owing to hysteresis. Vt_{sub} volumes during lung inflation for 50 cc volume stay with the right side of the curve within inflation of the curve; but during deflation, when the lung is already recruited, the same volume is moved at a lower pressure. For instance, at Vt_{sub} delivery at 600 cc during deflation (on left graph) is moved at a pressure of about 27, whereas during inflation Vt_{sub} delivery at 600 cc (on the right hand side) it took a pressure of about 36. This property of the lung indicates a need to have the ability to obtain compliance measurement while maintaining actual mechanical position of the lung which avoids yielding erroneous measurements. This property of lung mechanics was first published by J. Mead in the Journal of Applied Physiology in the mid-1950s.

Changes in dynamic compliance due to partially obstructed artificial airways or changes in lung resistance can be differentiated using pressure monitoring through the suction catheter during advancement and withdrawal through the airway as described above. A repeatable sudden change would identify a partial artificial obstruction necessitating appropriate intervention. A change in the ratio of the carinal pressure swing to the ventilator pressure swing would indicate a change in lung static compliance or lung resistance. This could be further differentiated through the occlusive static compliance measurement previously described. Corrective measures could then be determined.

The device allows for quick, easy determination of optimal HFV manipulation while avoiding the negative consequences of currently “blind” ventilator management. Furthermore, high frequency jet ventilation (HFJV) has also been shown to be most effective when delivered with positive end expiratory pressure (PEEP) set above the lower inflection of the lung which could be identified with this device. Lower and upper inflection points can be determined using this device, thereby allowing ventilator settings to be customized for a patient in high frequency percussive ventilation (HFPV) as well as conventional ventilation modes.

Thus, a device for secretion removal, manual ventilation and determination of in vivo lung mechanics without HFV circuit disconnection has been described. While specific isolation methods have been described above, certain modifications to the valve system chosen may be made while remaining within the broadest interpretation of the claims.

Although the present invention has been described in detail, those skilled in the art should understand that they can make various changes, substitutions and alterations herein without departing from the spirit and scope of the invention in its broadest form.

What is claimed is:

1. An artificial airway obstruction and pulmonary mechanics measuring device, comprising:
a rigid body chamber having a pressure measuring port and a medical ventilator port;
a pressure measuring device coupled to said pressure measuring port; and
a ventilator port occlusion device coupleable to said medical ventilator port.

2. The measuring device as recited in claim 1 wherein said rigid body chamber further comprises an artificial airway port.

3. The measuring device as recited in claim 1 wherein said rigid body chamber further comprises an air injection port.

4. The measuring device as recited in claim 1 wherein said ventilator port occlusion device comprises a pliable plunger slidably coupled to said rigid body chamber.

5. The measuring device as recited in claim 4 wherein said slidable pliable plunger comprises circumferentially-segmented locking lugs and said rigid body chamber further comprises complementary circumferentially-segmented locking lugs therewithin.

6. The measuring device as recited in claim 1 wherein said pressure measuring device comprises a flexible balloon catheter and said ventilator port occlusion device comprises an inflatable/deflatable cuff coupled around an end of said flexible balloon catheter.

7. The measuring device as recited in claim 1 wherein said ventilator port occlusion device comprises a valve.

8. The measuring device as recited in claim 7 wherein said valve is a rotary valve.

9. The measuring device as recited in claim 7 wherein said valve is a gate valve.

10. The measuring device as recited in claim 7 wherein said valve is a ball valve.

11. A method of manufacturing an artificial airway obstruction and pulmonary mechanics measuring device, comprising:
forming a rigid body chamber having a pressure measuring port and a medical ventilator port;
coupling a pressure measuring device to said pressure measuring port; and
coupling a ventilator port occlusion device to said medical ventilator port.

12. The method as recited in claim 11 wherein forming includes forming an artificial airway port in said rigid body chamber.

13. The method as recited in claim 11 wherein forming includes forming an air injection port in said rigid body chamber.

14. The method as recited in claim 11 wherein coupling said ventilator port occlusion device comprises slidably coupling a pliable plunger to said rigid body chamber.

15. The method as recited in claim 14 wherein said pliable plunger comprises segmented locking lugs and said rigid body chamber further comprises complementary segmented locking lugs therewithin.

16. The method as recited in claim 11 wherein said pressure measuring device comprises a flexible balloon catheter and said ventilator port occlusion device comprises coupling an inflatable/deflatable cuff around an end of said flexible balloon catheter.

17. The method as recited in claim 11 wherein coupling said ventilator port occlusion device comprises coupling a valve to said medical ventilator port.

18. The method as recited in claim 17 wherein coupling said valve to said medical ventilator port includes coupling a rotary valve to said medical ventilator port.

19. The method as recited in claim 17 wherein coupling said valve to said medical ventilator port includes coupling a gate valve to said medical ventilator port.

20. The method as recited in claim 17 wherein coupling said valve to said medical ventilator port includes coupling a ball valve to said medical ventilator port.
